(19) United States
(12) Patent Application Publication
(10) Pub. No.: US 2019/0381329 A1
(43) Pub. Date: Dec. 19, 2019

(54) SYSTEM AND METHOD FOR UNIPHASIC AND BIPHASIC SHOCK INVERSION TIME DOMAIN SHIFTING FOR SHOCK ENERGY VECTORING IN TRANSVENOUS AND SUBCUTANEOUS DEFIBRILLATORS WITH TWO OR MORE SHOCK VECTORS

(71) Applicant: NewPace Ltd., Caesarea (IL)

(72) Inventors: Robert Fishel, Delray Beach, FL (US); Moty Mocha, Beit Dagan (IL)

(21) Appl. No.: 16/478,857

(22) PCT Filed: Feb. 23, 2018
(86) PCT No.: PCT/IL2018/050212

§ 371 Date: Jul. 18, 2019

Related U.S. Application Data
(60) Provisional application No. 62/462,367, filed on Feb. 23, 2017.

(51) Int. Cl. A61N 1/39; A61N 1/05

(52) U.S. Cl. CPC A61N 1/3906; A61N 1/0504; A61N 1/375; A61N 1/3956; A61N 1/3918

(57) ABSTRACT

Method for truncating and summating shock vector energy between at least two shock vectors in a defibrillator, including the procedures of applying at least two biphasic defibrillating shock vectors simultaneously via at least two electrode sets until a voltage inversion point, terminating at least a first one of the biphasic defibrillating shock vectors at the voltage inversion point, and directing a remaining energy of the first one of the biphasic defibrillating shock vectors to a second phase of at least a second one of the biphasic defibrillating shock vectors.

### CROSS REFERENCE TO RELATED APPLICATIONS

This application is a 371 application from international patent application No. PCT/IL2018/050212, which claims priority to U.S. provisional patent application No. 62/462,367 filed Feb. 23, 2017, which is incorporated herein by reference in its entirety.

### FIELD OF THE DISCLOSED TECHNIQUE

The disclosed technique relates to defibrillators, in general, and to methods and systems for shifting the shock energy between shocks vectors in a defibrillator, in particular.

### BACKGROUND OF THE DISCLOSED TECHNIQUE

Defibrillators are heart devices which provide high energy shocks to the heart to reset the heart's electrical system and restore a normal cardiac rhythm in patients suffering from various types of arrhythmias involving heart rhythm disorders. High energy shocks to the heart have shown to restore normal cardiac rhythm in heart rhythm disorders such as ventricular tachycardia, ventricular fibrillation, atrial fibrillation and other types of rapid atrial and ventricular cardiac arrhythmias. Various types of defibrillators exist depending on their placement in or on the body, such as external defibrillators, transvenous defibrillators and subcutaneous defibrillators.

Defibrillators have may one or more shock vectors such that high energy shocks can be directed to one or more areas of the heart. Medical research has shown that the amount of energy required for effective defibrillation (i.e. to restore normal cardiac rhythm in a heart suffering from a heart rhythm disorder), also known as the defibrillating threshold (herein abbreviated DFT) has been shown to be lower if more than one high energy vector is used in a defibrillating shock.

Reference is now made to FIG. 1A, which is a schematic illustration of a transvenous defibrillator showing two shock vectors, generally referenced 10, as is known in the prior art. FIG. 1A shows a transvenous defibrillator 12, also referred as an intravenous defibrillator, endocardial defibrillator or implantable cardioverter defibrillator (herein abbreviated ICD) placed inside a human patient (not labeled) with a heart 24. Transvenous defibrillator 12 includes a can 14, a first lead 16, a second lead 18 and a third lead 20. Each of first lead 16, second lead 18 and third lead 20 is coupled with can 14. First lead 16 and second lead 18 each include a respective high voltage electrode 22A and 22B which can be used for delivering and receiving a high energy shock. Can 14 may also be equipped with an internal high voltage electrode (not shown). Transvenous defibrillator 12 represents an example of an FDA-approved transvenous defibrillator. As shown, can 14 is positioned in the left side of the chest of the patient, first lead 16 is positioned in the superior vena cava (not labeled) of heart 24 and second lead 18 is positioned in the right ventricle (not labeled) of heart 24. Third lead 20 may be positioned in the right atrium (not labeled) of heart 24 and is optional in transvenous defibrillator 12.

In transvenous defibrillator 12, a high energy shock can be delivered between any two high voltage electrodes as a shock vector. Transvenous defibrillator 12 includes three high voltage electrodes which are can 14, first lead 16 and second lead 18. As shown in FIG. 1A, a first shock vector 26A is provided between high voltage electrode 22B and high voltage electrode 22A whereas a second shock vector 26B is provided between high voltage electrode 22B and can 14. The shock vectors shown in FIG. 1A are a typical configuration for treating heart rhythm disorders. As shown in FIG. 1A, two pathways for electrical energy are generated that pass through heart 24. This configuration, as opposed to one which only includes a single shock vector, allows for a larger electrical field to be generated by transvenous defibrillator 12 to encompass a larger mass of fibrillating myocardium (not shown) of heart 24 and thus lead to effective defibrillation. Transvenous defibrillator 12 can be configured in other ways to provide more than two shock vectors.

Reference is now made to FIG. 1B, which is a schematic illustration of a subcutaneous defibrillator showing two shock vectors, generally referenced 50, as is known in the prior art. FIG. 1B shows a subcutaneous defibrillator 52 placed inside a human patient (not labeled) around a heart 58. As opposed to transvenous defibrillator 12 (FIG. 1A) in which the leads and electrode are placed within the heart, in subcutaneous defibrillator 52, all leads and electrodes are positioned around the heart subcutaneously. Subcutaneous defibrillator 52 includes four electrodes 54A, 54B, 54C and 54D and two sensors 56A and 56B. Electrodes 54A-54D can be used to deliver and receive high energy shocks whereas sensors 56A-56B can be used to detect the electrical activity of heart 58. Subcutaneous defibrillator 52 is shown schematically in FIG. 1B. More detailed descriptions of examples of subcutaneous defibrillator 52 can be found in US patent application publication no. US 2015/0343228 A1 to Strommer et al. and PCT international publication number WO 2016/038599 A1 to NewPace Ltd. As shown, one end of subcutaneous defibrillator 52 is placed over the anterior right side of heart 58 adjacent to the sternum (not labeled) whereas the other end of subcutaneous defibrillator 52 is placed over the posterior left side of heart 58, closer to the back (not labeled). The body (not labeled) of subcutaneous defibrillator 52 is positioned around heart 58 with a significant portion of the body placed in the abdominal region of the patient.

Given that subcutaneous defibrillator 52 includes four electrodes, various shock vectors can be transmitted through heart 58 to treat a heart rhythm disorder. For example, as shown in FIG. 1B, a first shock vector 60A is provided between electrode 54C to electrode 54A, providing an electric shock to the left ventricle and right atrium of heart 58, and a second shock vector 60B is provided between electrode 54B to electrode 54D, providing an electric shock to the right ventricle and left atrium of heart 58. Other shock vector configurations in subcutaneous defibrillator 52 are possible including ones that involve two, three or more shock vectors. It is noted as well that in subcutaneous defibrillators, higher energy shocks may be required as compared to transvenous defibrillators due to the increased distance between the electrodes (which are positioned subcutaneously) and the fibrillating myocardium (not shown) of heart 58.

Defibrillators commonly deliver high energy shocks to the heart via a uniphasic (also known as a monophasic) waveform or a biphasic waveform. Reference is now made to FIG. 2A, which is a graph showing the waveform energy of a biphasic shock vector used in a defibrillator, generally referenced 80, as is known in the prior art. It is noted that the waveform shown in FIG. 2A can be used in any type of defibrillator, whether external, transvenous or subcutaneous. Graph 80 includes an x-axis 82 representing time in milliseconds and a y-axis 84 representing voltage in volts. As shown, a curve 86 represents the change in voltage over time for a typical biphasic waveform. Typical biphasic waveforms may last between 15-20 milliseconds, delivering an initial high energy shock of approximately 1750 volts. Biphasic waveforms are named as such since they include two phases. Initially the shock vector is applied via a positive voltage, as shown by a curve section 88A. Since the shock vector is usually delivered by discharging a capacitor in the defibrillator (not shown), the amount of applied voltage begins to drop immediately after the initial discharge of the capacitor, as shown in curve section 88A. At approximately the middle of the delivery of the shock vector time wise, the shock vector is truncated by inverting the polarity of the voltage of the delivered shock vector. As shown at around 12 milliseconds, curve 86 changes polarity such that the remainder of the delivered shock vector is delivered using a negative voltage, shown as a curve section 88B. The total amount of energy delivered to the heart via the high energy shock is represented by the integral of curve 86, namely the area under curve 86, which includes the area under curve section 88A and the area under curve section 88B, represented respectively as areas 90A and 90B. The polarity of the voltage of the shock vector determines which direction the shock vector is delivered between a set of electrodes. In the case of a biphasic waveform, half the shock vector is delivered in one direction whereas the other half of the shock vector is delivered in the reverse direction. For example, with reference to FIG. 1B, a biphasic shock vector between electrodes 54B (FIG. 1B) and 54D (FIG. 1B) would mean that half the shock vector would be sent from electrode 54B to electrode 54D and when the polarity of the voltage is changed, the other half of the shock vector would be sent from electrode 54D to electrode 54B, in the reverse direction.

Reference is now made to FIG. 2B, which is a graph showing the waveform energy of a uniphasic shock vector used in a defibrillator, generally referenced 100, as is known in the prior art. It is noted that the waveform shown in FIG. 2B can be used in any type of defibrillator, whether external, transvenous or subcutaneous. Graph 100 includes an x-axis 102 representing time in milliseconds and a y-axis 104 representing voltage in volts. As shown, a curve 106 represents the change in voltage over time for a typical uniphasic waveform. Typical uniphasic waveforms may also last between 15-20 milliseconds, delivering an initial high energy shock of approximately 2000 volts. Uniphasic waveforms are named as such since they include a single phase applied via a positive voltage, as shown by curve 106. Since the shock vector is usually delivered by discharging a capacitor in the defibrillator (not shown), the amount of applied voltage begins to drop immediately after the initial discharge of the capacitor. As opposed to a biphasic waveform, a uniphasic waveform is only applied in one direction across a set of electrodes. Like a biphasic waveform, the total amount of energy delivered to the heart via the high energy shock is represented by the integral of curve 106, namely the area under curve 106, which includes areas 108 and 110. Area 108 represents the majority of the energy delivered to the heart via the uniphasic waveform, with a small amount of energy as shown in area 110 being delivered via a change in polarity of the voltage of the shock vector. For example, with reference to FIG. 1B, a uniphasic shock vector between electrodes 54B (FIG. 1B) and 54D (FIG. 1B) would mean that the shock vector would be sent from electrode 54B to electrode 54D in one direction.

In a defibrillator with two shock vectors, such as shown above in FIGS. 1A and 1B, the energy of the shock vectors is delivered by a single set of capacitors that discharge into both sets of electrodes simultaneously. For example, in FIG. 1A the energy for both of the shock vectors (first shock vector 26A and second shock vector 26B) is delivered simultaneously to high voltage electrode 22B and in FIG. 1B the energy for both of the shock vectors (first shock vector 60A and second shock vector 60B) is delivered simultaneously to electrodes 54B and 54C. Each shock vector will typically have a markedly different impedance depending on the actual path the shock vector travels from one electrode to another. In general, a higher current of electricity and thus more total power will preferentially pass through the shock vector with the lower impedance, thereby resulting in a disproportionate and asymmetric amount of energy being delivered to different regions of the heart by each shock vector. The relative amount of energy passing through each shock vector is determined by an individual patient's anatomy and the specific placement of the electrodes of the defibrillator in or around the patient's heart. Once the electrodes of a defibrillator are in place, the asymmetry in energy delivery to the heart between each of the two vectors cannot be altered unless the physical position of the electrodes of the defibrillator and/or the anatomy of the patient changes. The shunting of energy between two or more shock vectors will result in a differently shaped waveform energy curve for each shock vector of electrical energy passing through the heart. As mentioned above, the shock vector of highest impedance will result in a waveform that delivers little energy to the heart. Should too much current either be shunted to regions outside of the heart or too little current reach an area of critically fibrillating myocardium, a patient may fail to be defibrillated at a given energy level or may require higher than optimal energy levels to be defibrillated effectively. It may be that many shock vectors delivered with state of the art dual shock vector defibrillators ultimately fail to deliver maximum utilization of their shock energy due to wasted energy being shunted to regions of the body that do not require defibrillation.

The problem described above can be particularly serious in patients fitted with a subcutaneous defibrillator with an active electrical segment (such as described in PCT international publication number WO 2016/038599 A1 and shown schematically in FIG. 1B). In such a configuration, a first shock vector may pass between an electrode positioned near the left superior para-sternal region and an electrode (or an active electrical segment) positioned in the upper abdomen (such as between electrodes 54D and 54B (both in FIG. 1B)) and a second shock vector may pass between the electrode placed in the left superior para-sternal region and an electrode (or high voltage lead) positioned near the lateral portion or back of a patient (such as between electrodes 54D and 54C (both in FIG. 1B)). The first shock vector in this configuration will typically have substantially lower impedance than the second shock vector since the first shock vector will pass through the front of the chest to the patient's right ventricle whereas the second shock vector will pass through the thorax and lungs of the patient before reaching the patient's left ventricle. The first shock vector will pass a large amount of energy across the front chest and capture the right ventricle however the second shock vector will have higher impedance and pass less energy to the left ventricle since it will need to pass through a larger region of the thorax and lungs to capture the left ventricle. Both the first shock vector and the second shock vector are critical for successful defibrillation however the second shock vector will deliver lower total amounts of energy to the left ventricle given the higher impedance path of current flow, thus leading to an asymmetry in the amounts of energy delivered by the two shock vectors. In a subcutaneous defibrillator, such a configuration will result in higher energy requirements and DFTs for the second shock vector than might be required for the first shock vector, thus reducing the battery life of such a defibrillator and also applying more electricity than might be necessary for effectively defibrillating a patient's heart.

Defibrillators delivering two or more shock vectors with variable waveform energy curves are known in the art. US patent application publication no. 2013/0158614 to Azar et al., assigned to Smartwave Medical Ltd. and entitled “Pulse Parameters and Electrode Configurations for Reducing Patient Discomfort from Defibrillation” is directed to defibrillation systems and methods, and more specifically pulse parameters and electrode configurations for reducing patient discomfort in implantable defibrillators. The implantable defibrillator has an electrode lead system and at least one sensor for sensing a heart condition and emitting a condition signal. The defibrillator also has a controller in communication with the sensor and configured to determine from the condition signal whether the heart is fibrillating and emitting a command signal if fibrillation is detected. The defibrillator further has a voltage generator communicating with the controller and the electrode system to communicate at least one defibrillation pulse to the electrode system. The defibrillation pulse includes at least one pulse having a voltage greater than 80 volts and a time duration up to 1000 microseconds. The pulse may be delivered to an atrium and/or a ventricle of the heart and have an electric field strength between 100 and 700 volts per centimeter. The pulse may deliver a total amount of energy to the heart that is less than 2 Joules. The voltage of the pulse may be between 80 and 3000 volts and/or 600 volts or greater. The sensor may be an electrode of the electrode lead system.

US patent application publication no. 2002/0138104 to Brewer et al., assigned to SurVivaLink Corporation and entitled “Method and Apparatus for Delivering a Biphasic Defibrillation Pulse with Variable Energy” is directed to an apparatus and method for determining an optimal transchest external defibrillation waveform that provides for variable energy in the first or second phase of a biphasic waveform that, when applied through a plurality of electrodes positioned on a patient's torso, will produce a desired response in the patient's cardiac cell membranes. The method includes the steps of providing a quantitative model of a defibrillator circuit for producing external defibrillation waveforms, a quantitative model of a patient including a chest component, a heart component, a cell membrane component and a quantitative description of the desired cardiac membrane response function. Finally, a quantitative description of a transchest external defibrillation waveform that will produce the desired cardiac membrane response function is computed. The computation is made as a function of the desired cardiac membrane response function, the patient model and the defibrillator circuit model. The defibrillation waveform is tailored and reformed such that a second phase of a biphasic defibrillation waveform relative to a first phase of the waveform is based upon the computation. The computation is based on the first phase cell response and is used to determine the desired second phase waveform.

US patent application publication no. 2015/0119948 to Trayanova et al. and entitled “Method for Low Voltage Defibrillation with Far-Field Stimuli of Variable Timings Based on Feedback from the Heart” is directed to a method for cardiac defibrillation, especially low-voltage defibrillation, in a subject. The method includes converting fibrillation into tachycardia and using feedback or an estimation thereof from the heart to time stimuli to occur when large amounts of tissue are excitable in the heart of the subject. The resultant tachycardia can then be terminated using a tachycardia termination protocol known to or conceivable by one of skill in the art. A cardiac signal is obtained using electrocardiography. A target time for applying stimulation to the heart of the subject is estimated, the target time being approximately the maximum amount of excitable tissue of the heart of the subject. Far-field stimulation is applied using at least one of a defibrillator, an internal cardioverter-defibrillator or another implanted defibrillation device. Each far-field stimulus has a variable time of application determined using feedback or an estimate thereof from the heart of the subject. The first stimulation can take the form of multiple series of stimuli. The first far-field stimulation takes the form of one of either a monophasic or a biphasic shock, or an alternate shock waveform. Since the proposed defibrillation method requires less energy than current approaches using single biphasic stimulus, defibrillator battery life is improvable or battery size decreasable. The method also reduces pain and cellular damage resultant from traditional defibrillation.

US patent application publication no. 2014/0257425 to Arcot-Krishnamurthy et al., assigned to Cardiac Pacemakers Inc. and entitled “Hypertension Therapy Device with Longevity Management” is directed to a system and methods for programming and delivering electrical stimulation to treat hypertension. An ambulatory stimulator system, such as an implantable medical device, can receive a power-saving command and deliver the electrical stimulation to a target site in a patient according to one or more simulation parameters including a therapy on-off pattern. Stimulation with therapy on-off patterns can reduce the power consumption while maintaining the anti-hypertension therapy efficacy. The ambulatory stimulator system can include one or more of a physiologic response detector, a patient status detector or a battery longevity detector. The power-saving command can be generated using one or more of the detected physiologic signal, the patient status, or the information about the battery longevity. The system can have a memory configured to store one or more stimulation parameters including a therapy-on period during which the stimulation pulses are programmed to be delivered, a therapy-off period during which no stimulation pulse is programmed to be delivered, and a therapy on-off pattern including a combination of a sequence of therapy-on periods with variable durations and a sequence of therapy-off periods with variable durations. The therapy on-off patterns can be used to conserve the power consumption for arterial hypertension therapy. A control circuit can be configured to receive a power-saving command, time one or both of the therapy-on period and the therapy-off period, and schedule the delivery of the stimulation pulses to a target site according to the therapy on-off pattern in response to a power-saving command.

US patent application publication no. 2002/0035382 to Rubin et al., assigned to Intermedics Inc. and entitled “Methods and Apparatus for Treating Fibrillation and Creating Defibrillation Waveforms” is directed to methods and an apparatus for treating fibrillation utilizing biphasic waveforms. A cardiac stimulator includes a defibrillation circuit that uses a pulse width modulated capacitive discharge to generate various biphasic waveforms, one or more of which may be delivered to the heart to treat the fibrillation. The biphasic defibrillation waveform can include a positive voltage phase beginning at about zero volts and having an initial positive voltage magnitude greater than zero volts. The positive voltage phase has a first positively sloped portion extending from the initial positive voltage magnitude to a maximum positive voltage magnitude greater than the initial positive voltage magnitude. A negative voltage phase has an initial maximum negative voltage magnitude less than zero volts extending from the maximum positive voltage magnitude of the positive voltage phase. The negative voltage phase has a second positively sloped portion extending from the initial maximum negative voltage magnitude to a terminal negative voltage magnitude greater than the initial maximum negative voltage magnitude. The biphasic defibrillation waveform can be provided using a defibrillation waveform generator that includes an arrhythmia detector adapted to be coupled to a heart, the arrhythmia detector delivering a detection signal in response to detecting fibrillation in the heart. The defibrillation waveform generator also includes a charging circuit coupled to a capacitor, the charging circuit charging the capacitor to a given voltage and a controller operably coupled to the arrhythmia detector to receive the detection signal, the controller delivering a first control signal, a second control signal, and a third control signal in response to receiving the detection signal. The defibrillation waveform generator further includes a voltage-to-frequency convertor coupled to the controller to receive the first control signal, the voltage-to-frequency convertor delivering a frequency signal having a frequency correlative to the first control signal and a pulse width modulator coupled to the controller to receive the second control signal and coupled to the voltage-to-frequency convertor to receive the frequency signal. The pulse width modulator delivers a pulse width modulated signal having a frequency correlative to the frequency signal and having a duty cycle correlative to the second control signal. The defibrillation waveform generator finally includes a switching circuit adapted to be coupled between the capacitor and the heart, the switching circuit being coupled to the controller to receive the third control signal and to the pulse width modulator to receive the pulse width modulated signal, the switching circuit controllably discharging the capacitor across the heart to deliver a defibrillation waveform in response to the third control signal and the pulse width modulated signal.

### SUMMARY OF THE DISCLOSED TECHNIQUE

It is an object of the disclosed technique to provide a novel method and system for shifting the shock energy between shocks vectors in a defibrillator which overcomes the disadvantages of the prior art.

In accordance with the disclosed technique, there is thus provided a method for truncating and summating shock vector energy between at least two shock vectors in a defibrillator. The method includes the procedures of applying at least two biphasic defibrillating shock vectors simultaneously via at least two electrode sets until a voltage inversion point and terminating at least a first one of the biphasic defibrillating shock vectors at the voltage inversion point. The method also includes the procedure of directing a remaining energy of the first one of the biphasic defibrillating shock vectors to a second phase of at least a second one of the biphasic defibrillating shock vectors.

In accordance with another aspect of the disclosed technique, there is thus provided a subcutaneous defibrillator for truncating and summating at least two biphasic defibrillating shock vectors, including a body, a plurality of electrodes and a plurality of sensors. The plurality of electrodes are positioned on the body and are for applying the biphasic defibrillating shock vectors and the plurality of sensors are positioned on the body for detecting arrhythmias. The body includes at least one capacitor, a processor and at least one battery. The processor is coupled with the capacitor and the battery is coupled with the capacitor and the processor. The capacitor is for storing charge for providing the biphasic defibrillating shock vectors and the battery is for charging the capacitor and for providing energy to operate the processor. The electrodes apply at least a first one of the biphasic defibrillating shock vectors and at least a second one of the biphasic defibrillating shock vectors simultaneously until a voltage inversion point. The processor terminates the first one of the biphasic defibrillating shock vectors at the voltage inversion point and directs a remaining energy of the first one of the biphasic defibrillating shock vectors to a second phase of the second one of the biphasic defibrillating shock vectors.

In accordance with a further aspect of the disclosed technique, there is thus provided a defibrillator for truncating and summating at least two biphasic defibrillating shock vectors. The defibrillator includes a can and a plurality of leads. The leads are coupled with the can and are for detecting arrhythmias. The can includes at least one capacitor, a processor and at least one battery. The processor is coupled with the capacitor and the battery is coupled with the capacitor and the processor. The capacitor is for storing charge for providing the biphasic defibrillating shock vectors and the battery is for charging the capacitor and for providing energy to operate the processor. The leads apply at least a first one of the biphasic defibrillating shock vectors and at least a second one of the biphasic defibrillating shock vectors simultaneously until a voltage inversion point. The processor terminates the first one of the biphasic defibrillating shock vectors at the voltage inversion point and directs a remaining energy of the first one of the biphasic defibrillating shock vectors to a second phase of the second one of the two biphasic defibrillating shock vectors.

### BRIEF DESCRIPTION OF THE DRAWINGS

The disclosed technique will be understood and appreciated more fully from the following detailed description taken in conjunction with the drawings in which:

FIG. 1A is a schematic illustration of a transvenous defibrillator showing two shock vectors, as is known in the prior art;

FIG. 1B is a schematic illustration of a subcutaneous defibrillator showing two shock vectors, as is known in the prior art;

FIG. 2A is a graph showing the waveform energy of a biphasic shock vector used in a defibrillator, as is known in the prior art;

FIG. 2B is a graph showing the waveform energy of a uniphasic shock vector used in a defibrillator, as is known in the prior art;

FIG. 3 is a graph showing current delivery and decay curves across different impedances in a defibrillator, constructed and operative in accordance with an embodiment of the disclosed technique;

FIG. 4 is a schematic illustration of a subcutaneous defibrillator showing two shock vectors, constructed and operative in accordance with another embodiment of the disclosed technique;

FIG. 5 is a set of graphs showing the truncation and summation of shock energy between two shock vectors, constructed and operative in accordance with a further embodiment of the disclosed technique;

FIG. 6 is a set of graphs showing different truncations and summations of shock energy between two shock vectors by displacement of an inversion point, constructed and operative in accordance with another embodiment of the disclosed technique;

FIG. 7 is a schematic illustration of defibrillators for truncating and summating shock energy between two shock vectors, constructed and operative in accordance with a further embodiment of the disclosed technique; and

FIG. 8 is a method for truncating and summating shock energy between two shock vectors, operative in accordance with another embodiment of the disclosed technique.

### DETAILED DESCRIPTION OF THE EMBODIMENTS

The disclosed technique overcomes the disadvantages of the prior art by providing a novel method and system for optimizing the energy delivered in defibrillators with two or more shock vectors. By optimizing the energy provided to each shock vector, problems relating to energy shunting in multi-vectorial defibrillation pulses can be minimized and lower DFTs can be achieved in external, transvenous and subcutaneous defibrillators. According to the disclosed technique, in defibrillators employing two or more biphasic shock vectors, the shock vector with the lower impedance is truncated at its inversion point such that it is made into a unipolar shock vector having only one phase (similar to a uniphasic shock vector) and terminated at its inversion point. The energy of the second phase of the shock vector with lower impedance is transferred and added to the shock vector with higher impedance. Thus, the remaining energy of the second phase of the shock vector with lower impedance is electronically switched from one set of electrodes and directed to another set of electrodes which deliver the shock vector of higher impedance. The biphasic shock vector of higher impedance now includes additional current and voltage from the other biphasic shock vector (now uniphasic) and thus provides an increase in deliverable energy via the shock vector with higher impedance. According to the disclosed technique, some of the energy being provided to an area of lower impedance in and around the heart is therefore shifted to an area of higher impedance in and around the heart, thus achieving an increase in symmetry in the total energy delivered to the heart by shock vectors having difference impedances.

Also according to the disclosed technique, further symmetry between the shock vectors can be achieved by moving the inversion point of the shock vector with lower impedance. In this manner, substantially full symmetry and balance of energy delivery between the shock vectors can be achieved. By moving the inversion point sooner in time, more energy can be transferred to the shock vector with higher impedance, whereas extending the inversion point in time will provide less energy transfer to the shock vector with higher impedance. According to the disclosed technique, a physician or clinician can optimize the delivery of energy to the two or more shock vectors of a defibrillator of a patient such that the DFT of the particular patient for achieving effective defibrillation is as low as possible. Lower overall DFTs (which by definition provide effective defibrillation) are better for patient comfort and can increase the battery life of an implanted defibrillator, whether implanted transvenously or subcutaneously. As compared with the prior art, the disclosed technique enables the energy delivery in defibrillators to be dynamically shifted and optimized between two or more shock vectors.

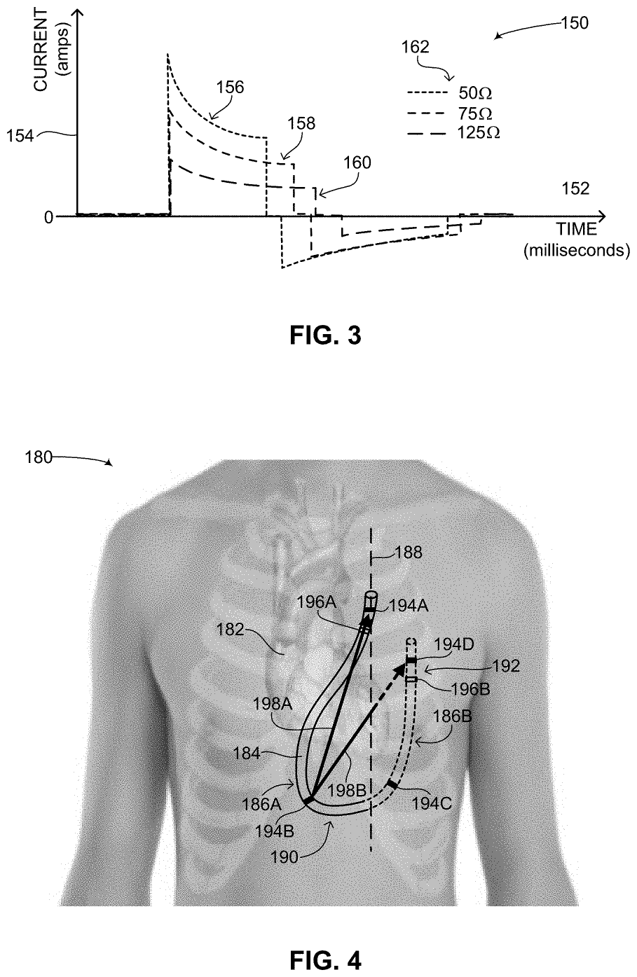

Reference is now made to FIG. 3, which is a graph showing current delivery and decay curves across different impedances in a defibrillator, generally referenced 150, constructed and operative in accordance with an embodiment of the disclosed technique. Graph 150 includes an x-axis 152 showing time in milliseconds and a y-axis 154 showing current in amperes (or for short, amps). Graph 150 shows the distribution of current over time for three different waveforms of shock vectors used in a defibrillator (not shown), each one representing a different amount of impedance as per a legend 162. A first waveform 156 shows the amount of current provided by the defibrillator where the shock vector experiences the least amount of impedance. A second waveform 158 shows a reduced amount of current provided by the defibrillator with an increase in impedance whereas a third waveform 160 shows a further reduced amount of current provided by the defibrillator with a further increase in impedance. First waveform 156 provides the most amount of energy in a shock vector to fibrillating myocardium whereas third waveform 160 provides the least amount of energy in a shock vector to fibrillating myocardium. As explained above, the particular placement of electrodes of an implantable defibrillator as well as the unique anatomy of a patient usually results in multi-vectorial pulses (i.e., defibrillators with two or more shock vectors) encountering different amounts of impedance as they cross the heart of a patient. Therefore typically, a patient being defibrillated by two or more shock vectors can experience a first shock vector similar to first waveform 156, which may defibrillate a first part of the heart (such as the right ventricle) effectively, whereas a second shock vector may be similar to third waveform 160, which will not defibrillate a second part of the heart (such as the left ventricle) effectively. According to the disclosed technique, the energy between two or more shock vectors can be distributed differently such that both shock vectors defibrillate effectively even if the path travelled by each shock vector has a different impedance as shown in graph 150.

Reference is now made to FIG. 4, which is a schematic illustration of a subcutaneous defibrillator showing two shock vectors, generally referenced 180, constructed and operative in accordance with another embodiment of the disclosed technique. As shown, subcutaneous defibrillator 180 is placed around a heart 182 of a patient (not labeled). Subcutaneous defibrillator 180 includes a body 184, a plurality of electrodes 194A-194D and a plurality of sensors 196A and 196B. High energy shocks can be provided by subcutaneous defibrillator 180 between any two of plurality of electrodes 194A-194D. A portion of body 184 of subcutaneous defibrillator 180 is placed anteriorly whereas another portion of body 184 is placed posteriorly in the back 192 of the patient. As shown, a dotted line 188 demarcates the anterior and posterior locations of subcutaneous defibrillator 180, where a portion 186A of body 184 is placed anterior to heart 182 and a portion 186B of body 184 is placed posterior to heart 182. Subcutaneous defibrillator 180 is placed around heart 182 such that a significant portion of it is placed in the abdominal region 190 of the patient. The positioning and functioning of an embodiment of subcutaneous defibrillator 180 is explained in more detail in PCT international publication number WO 2016/038599 A1.

An example of two shock vectors being provided to heart 182 is shown in FIG. 4. A first shock vector 198A is provided from electrode 194B to electrode 194A and a second shock vector 198B is provided from electrode 194B to electrode 194D. Other shock vector configurations are possible between different electrodes, including the possibility of more than two shock vectors, for example between electrode 194C and electrode 194A, between electrode 194D and electrode 194B and between electrode 194A and 194D. Both first shock vector 198A and second shock vector 198B are provided simultaneously to heart 182. In addition, first shock vector 198A and second shock vector 198B are biphasic shock vectors, meaning that during a first phase of both shock vectors, electrical current is passed from electrode 1948 to electrodes 194A and 194D respectively. During a second phase of both shock vectors, when the polarity of the voltage is inverted, electrical current is passed from electrodes 194A and 194D to electrode 194B respectively, in the opposite direction. Due to the placement of plurality of electrodes 194A-194D and the anatomy of the patient, first shock vector 198A will experience less impedance as compared with second shock vector 198B. Thus the right ventricle (not labeled) of heart 182 will most probably receive enough energy to effectively defibrillate whereas the left ventricle (not labeled) of heart 182 will most probably not receive enough energy to effectively defibrillate. This is due to the increased distance and cavities second shock vector 198B has to travel between electrodes as compared with first shock vector 198A.

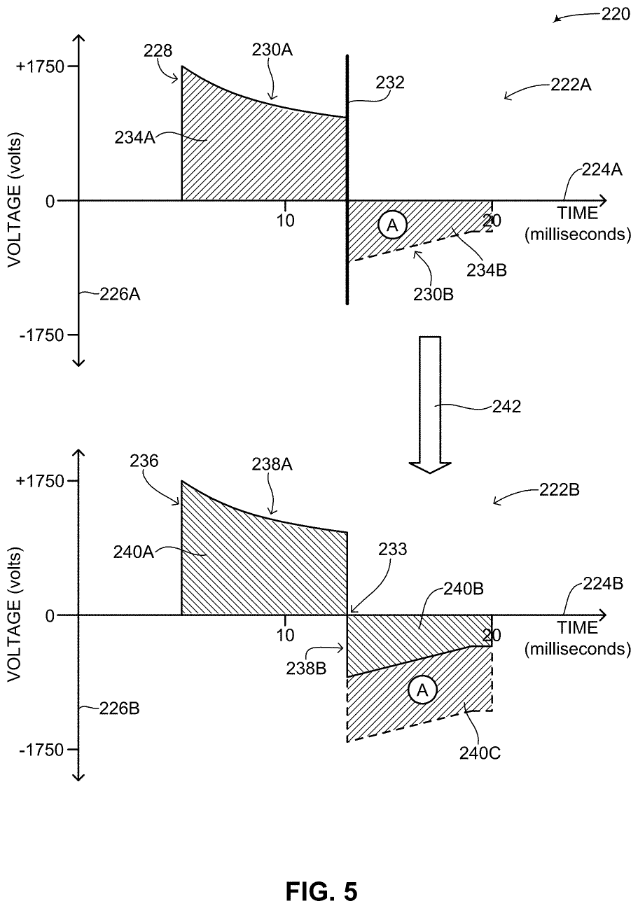

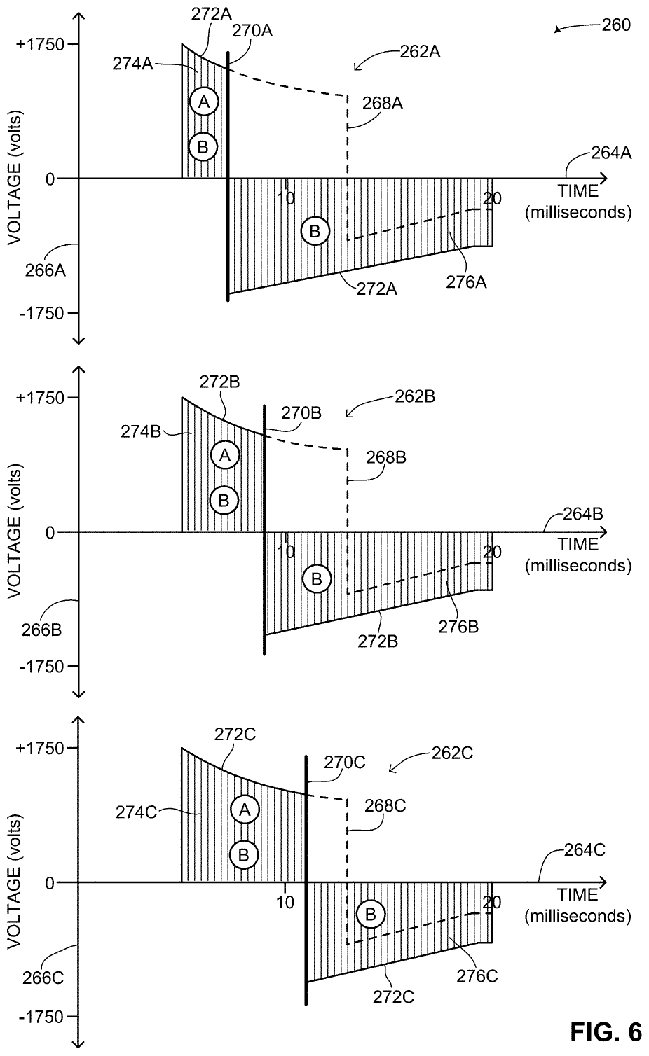

The imbalance in energy delivery between the two shock vectors in FIG. 4 can be balanced according to the disclosed technique as shown below and explained in FIGS. 5 and 6. Reference is now made to FIG. 5, which is a set of graphs showing the truncation and summation of shock energy between two shock vectors, generally referenced 220, constructed and operative in accordance with a further embodiment of the disclosed technique. A first graph 222A shows a waveform energy curve 228 of a first biphasic shock vector. First graph 222A includes an x-axis 224A showing time in microseconds and a y-axis 226A showing voltage in volts. As shown, the first biphasic shock vector includes two phases, a first phase 230A wherein the waveform energy is applied using a positive voltage and a second phase 230B wherein the waveform energy is applied using a negative voltage. The inversion point in time where the voltage of the applied waveform energy changes from positive to negative is shown via a line 232. As mentioned above, the area under waveform energy curve 228 represents the amount of energy delivered by the first biphasic shock vector. An area 234A in first phase 230A shows the energy delivered by the first phase of the biphasic shock vector whereas an area 234B in second phase 230B shows the energy delivered by the second phase of the biphasic shock vector.

A second graph 222B shows a waveform energy curve 236 of a second biphasic shock vector. Second graph 222B includes an x-axis 224B showing time in microseconds and a y-axis 226B showing voltage in volts. As shown, the second biphasic shock vector includes two phases, a first phase 238A wherein the waveform energy is applied using a positive voltage and a second phase 238B wherein the waveform energy is applied using a negative voltage. Like in waveform energy curve 228, an inversion point in time for waveform energy curve 236, shown via an arrow 233, is where the voltage of the applied waveform energy changes from positive to negative. As mentioned above, the area under waveform energy curve 236 represents the amount of energy delivered by the second biphasic shock vector. An area 240A in first phase 238A shows the energy delivered by the first phase of the biphasic shock vector whereas an area 240B in second phase 238B shows the energy delivered by the second phase of the biphasic shock vector.

First and second biphasic shock vectors, with their waveform energy curves as shown in graphs 222A and 222B, are applied simultaneously. According to the disclosed technique, the waveform of the first biphasic shock vector, as shown in graph 222A, is terminated at the inversion point shown by line 232 such that waveform energy curve 228 is effectively only a unipolar shock vector. The energy of second phase 230B, represented by area 234B and shown by the letter ‘A’ is directed, shown by an arrow 242, to the second biphasic shock vector, as shown in graph 222B as an addition area 240C to second phase 238B. Since both the first and second biphasic shock vectors are applied simultaneously, terminating the first biphasic shock vector at the end of its first phase and directing the remaining energy to the second biphasic shock vector effectively increases the amount of energy delivered by the second biphasic shock vector. The first biphasic shock vector is thus converted into a uniphasic shock vector whereas the second biphasic shock vector remains biphasic with an increase in energy in its second phase.

Referring back to the example shown in FIG. 4, using the disclosed technique, first shock vector 198A (FIG. 4) is thus unipolar and substantially uniphasic whereas second shock vector 1988 (FIG. 4) has increased energy in its second phase. Since the path travelled by first shock vector 198A has less impedance, less energy is required to effectively defibrillate. Since the path travelled by second shock vector 198B has more impedance, more energy is required to effectively defibrillate. According to the disclosed technique, more energy can be provided to second shock vector 198B to compensate for the increase in impedance in the path travelled by that shock vector without increasing the actual amount of energy delivered by subcutaneous defibrillator 180 (FIG. 4). This is achieved by terminating first shock vector 198A at its inversion point and transferring the remaining energy of first shock vector 198A to the electrodes delivering second shock vector 1988.

Reference is now made to FIG. 6, which is a set of graphs showing different truncations and summations of shock energy between two shock vectors by displacement of an inversion point, generally referenced 260, constructed and operative in accordance with another embodiment of the disclosed technique. FIG. 6 shows three different graphs 262A, 262B, 262C of waveform energy curves for shock vectors used in accordance with the disclosed technique. Each graph includes an x-axis showing times in milliseconds, respectively an x-axis 264A, an x-axis 264B and an x-axis 264C and a y-axis showing voltage in volts, respectively a y-axis 266A, a y-axis 266B and a y-axis 266C. For reference as well, each graph shows a waveform energy curve wherein the inversion point has not been displaced, respectively waveform energy curves 268A, 268B and 268C. As mentioned above in FIG. 5, more energy can be delivered to a shock vector traversing a path of higher impedance by terminating a first shock vector at its inversion point and transferring the remaining energy of the first shock vector to a second shock vector, which is the shock vector traversing the path of higher impedance, thereby increasing the energy of the second phase of the second shock vector. Since the first and second shock vectors are delivered simultaneously in a defibrillator, further distribution between the energy delivered by each shock vector can be achieved by displacing the inversion point wherein the first shock vector is terminated and its remaining energy is transferred to the second shock vector. Terminating the first phase of the first shock vector earlier in time results in greater energy being transferred to the second shock vector whereas terminating the first phase of the first shock vector later in time results in less energy being transferred to the second shock vector.

Graph 262A shows a waveform energy curve 272A truncated at an early inversion point 270A. An area 274A represents the energy of the first phase of waveform energy curve 272A whereas an area 276A represents the energy of the second phase of waveform energy curve 272A. Area 274A is designated with the letters ‘A’ and ‘B’ showing that the energy in area 274A is delivered by both a first shock vector (zone ‘A’) and a second shock vector (zone ‘B’). In accordance with the disclosed technique, the first shock vector is terminated and truncated at inversion point 270A and the remaining energy of the first shock vector, area 276A, is added, or summated, to the energy of the second shock vector. Area 276A is thus shown as zone ‘B’ to indicate that this energy from the first shock vector is added to the energy of the second shock vector.

Graph 2626 shows a waveform energy curve 2726 truncated at a later inversion point 2706 as compared with inversion point 270A. An area 2746 represents the energy of the first phase of waveform energy curve 2726 whereas an area 2766 represents the energy of the second phase of waveform energy curve 2726. Area 2746 is designated with the letters ‘A’ and ‘B’ showing that the energy in area 2746 is delivered by both a first shock vector (zone ‘A’) and a second shock vector (zone ‘B’). In accordance with the disclosed technique, the first shock vector is terminated and truncated at inversion point 2706 and the remaining energy of the first shock vector, area 2766, is added, or summated, to the energy of the second shock vector. Area 2766 is thus shown as zone ‘B’ to indicate that this energy from the first shock vector is added to the energy of the second shock vector.

Graph 262C shows a waveform energy curve 272C truncated at an even later inversion point 270C as compared with inversion points 270A and 270B. An area 274C represents the energy of the first phase of waveform energy curve 272C whereas an area 276C represents the energy of the second phase of waveform energy curve 272C. Area 274C is designated with the letters ‘A’ and ‘B’ showing that the energy in area 274C is delivered by both a first shock vector (zone ‘A’) and a second shock vector (zone ‘B’). In accordance with the disclosed technique, the first shock vector is terminated and truncated at inversion point 270C and the remaining energy of the first shock vector, area 276C, is added, or summated, to the energy of the second shock vector. Area 276C is thus shown as zone ‘B’ to indicate that this energy from the first shock vector is added to the energy of the second shock vector.

As shown in graphs 262A, 262B and 262C, the inversion point of the waveform energy curve can be shifted in the time domain to change when the first shock vector is truncated and its remaining energy is summated to the energy of the second shock vector. Graph 262A shows an early inversion point and thus a significant amount of energy transfer to the second shock vector whereas graph 262C shows a later inversion point and closer to the energy transfer shown above in FIG. 5. In all the examples shown in FIG. 6, the first shock vector is unipolar, as shown by letters ‘A’ and ‘B’ in areas 274A, 274B and 274C, with the remainder of the energy of the first shock vector being transferred to the second shock vector which remains a biphasic shock vector. Even though the energy provided by the first shock vector to zone A is also provided by the second shock vector to zone B during the first phase of both shock vectors, which may deliver more energy to a lower impedance path through the heart of a patient, the second phase of both shock vectors is only provided to zone B, to the second shock vector, thus increasing the total amount of energy transferred to the second shock vector which traverses a path of higher impedance. By moving the timing of the inversion point, more energy can be shunted to the shock vector which has to traverse a path of higher impedance (i.e., zone B), thus balancing the total energy delivered between zones A and B. As described below, the particular inversion point for a given patient is to be determined by the clinician based on the patient's anatomy and the actual placement of the electrodes of the defibrillator in the patient.

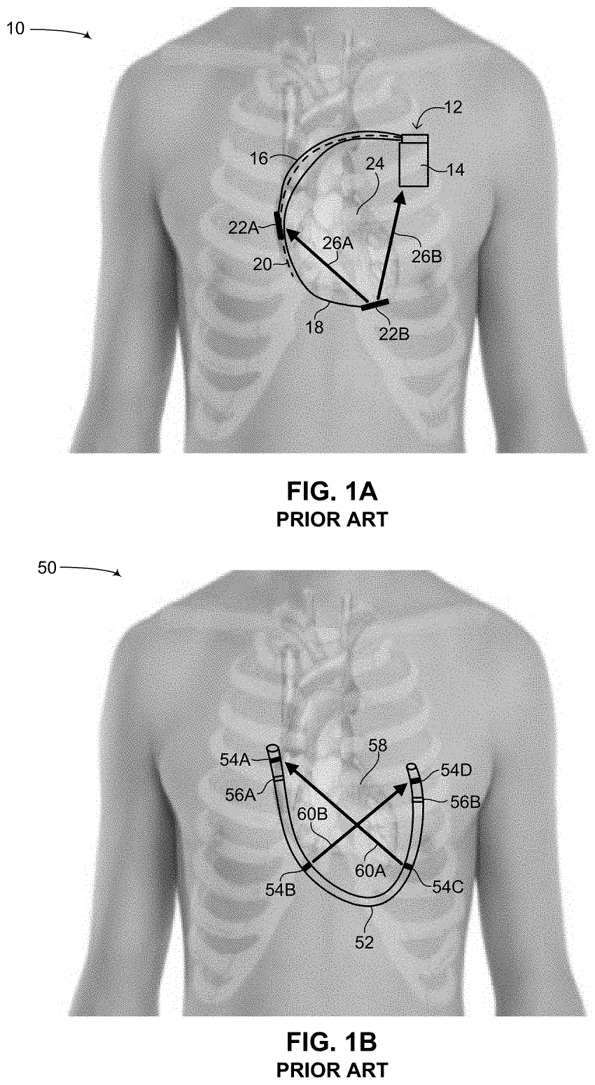

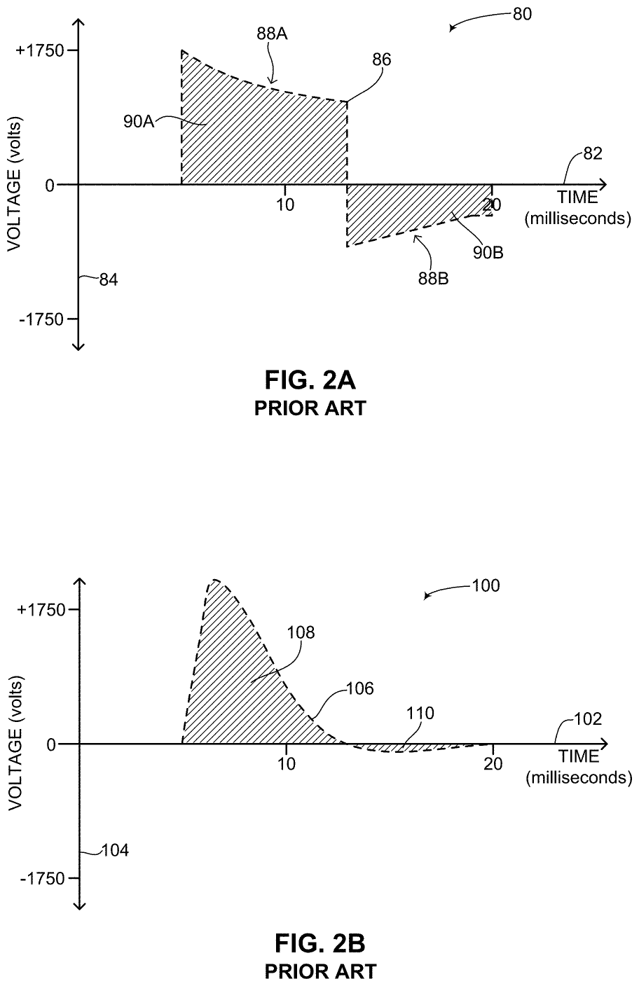

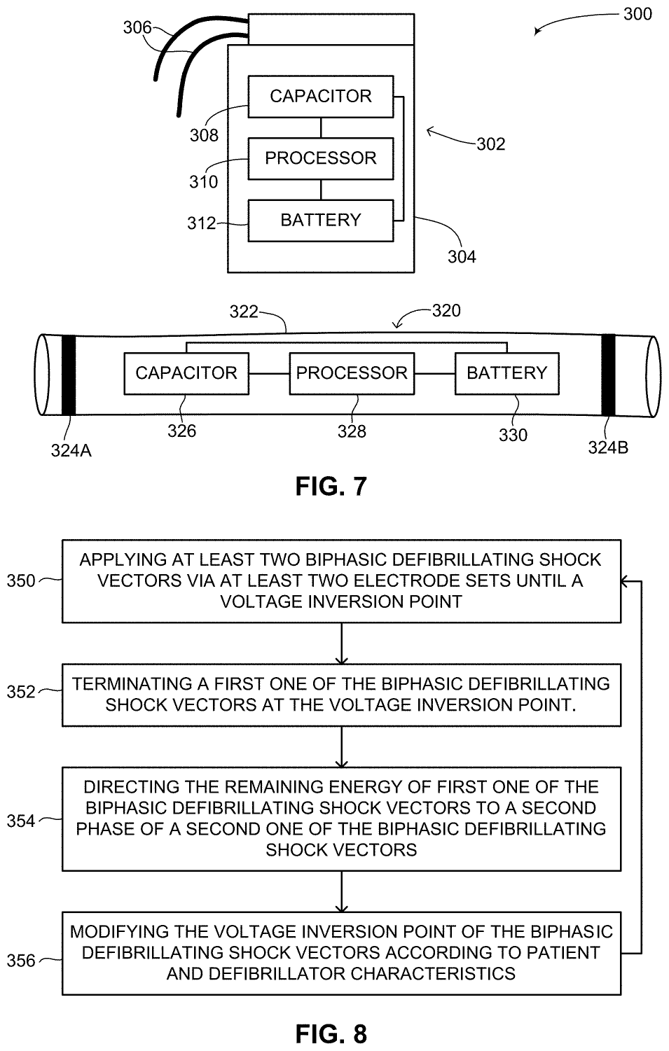

Reference is now made to FIG. 7, which is a schematic illustration of defibrillators for truncating and summating shock energy between two shock vectors, generally referenced 300, constructed and operative in accordance with a further embodiment of the disclosed technique. FIG. 7 shows a transvenous (or intravenous) defibrillator 302 as well as a subcutaneous defibrillator 320. Transvenous defibrillator 302 includes a can 304 and a plurality of leads 306, similar to transvenous defibrillator 12 (FIG. 1A). Can 304 includes a capacitor 308, a processor 310 and a battery 312. Processor 310 is coupled with capacitor 308 and battery 312 and capacitor 308 is also coupled directly with battery 312. Battery 312 is used to power processor 310 and to charge capacitor 308 with electrical charge. Plurality of leads 306 may include sensors (not shown) for detecting arrhythmias as well as electrodes (not shown) for applying electrical shocks to the heart of a patient. Processor 310 may include circuitry (not shown) for determining when an arrhythmia is occurring and for discharging electrical charge on capacitor 308 via plurality of leads 306. Given that transvenous defibrillator 302 includes a plurality of leads, transvenous defibrillator 302 can provide at least two or more shock vectors simultaneously to the heart of the patient. Processor 310 directs capacitor 308 to discharge equal amounts of charge as two separate shock vectors on different sets of electrodes (not shown) on the leads. Processor 310 can be programmed to apply a first shock vector as a unipolar or uniphasic shock vector and a second shock vector as a bipolar or biphasic shock vector. At the inversion point when processor 310 directs capacitor 308 to reverse the polarity of the discharged electricity to both shock vectors, the energy discharged to the electrodes delivering the first shock vector is truncated and shunted towards the electrodes delivering the second shock vector. It is noted that this function of the processor diverting the energy of the second phase of the first shock vector to the second shock vector may be made a programmable feature of processor 310 such that a clinician can decide to either have transvenous defibrillator 302 transmit two biphasic shock vectors, as in prior art defibrillators, or to transmit a truncated uniphasic shock vector and summated biphasic shock vector, as described above in FIG. 5.

In addition, processor 310 may be programmed with an option for changing or shifting the inversion point of the applied shock vectors. As explained above in FIG. 6, shifting the inversion point according to the disclosed technique can alter the energy distribution applied by each of two or more shock vectors applied to a patient's heart. Dynamically changing the inversion point in the time domain utilizing the disclosed technique will result in changes in the relative energy delivered between the two shock vectors. Processor 310 may include Bluetooth and/or infrared technology (not shown) for enabling a clinician to communicate with processor 310 via software and to either modify the inversion point or to turn the truncating/summating option of the two shock vectors on or off.

Subcutaneous defibrillator 320 includes a body 322 and a plurality of electrodes 324A and 324B, similar to subcutaneous defibrillator 52 (FIG. 1B). Body 322 includes a capacitor 326, a processor 328 and a battery 330. Processor 328 is coupled with capacitor 326 and battery 330 and capacitor 326 is also coupled directly with battery 330. As described above regarding transvenous defibrillator 302, battery 330 is used to power processor 328 and to charge capacitor 326 with electrical charge. Plurality of electrodes 324A and 324B can apply electrical shocks to the heart of a patient and may also include sensors (not shown) for detecting arrhythmias. Processor 328 may include circuitry (not shown) for determining when an arrhythmia is occurring and for discharging electrical charge on capacitor 326 via plurality of electrodes 324A and 324B. Given that subcutaneous defibrillator 320 includes a plurality of electrodes, subcutaneous defibrillator 320 can provide at least two or more shock vectors simultaneously to the heart of the patient. Processor 328 directs capacitor 326 to discharge equal amounts of charge as two separate shock vectors (not shown) on different sets of electrodes. Processor 328 can be programmed to apply a first shock vector as a unipolar or uniphasic shock vector and a second shock vector as a bipolar or biphasic shock vector. At the inversion point when processor 328 directs capacitor 326 to reverse the polarity of the discharged electricity to both shock vectors, the energy discharged to the electrodes delivering the first shock vector is truncated and shunted towards the electrodes delivering the second shock vector. It is noted that this function of the processor diverting the energy of the second phase of the first shock vector to the second shock vector may be made a programmable feature of processor 328 such that a clinician can decide to either have subcutaneous defibrillator 320 transmit two biphasic shock vectors, as in prior art defibrillators, or to transmit a truncated uniphasic shock vector and summated biphasic shock vector, as described above in FIG. 5. Using the disclosed technique in the case of a subcutaneous defibrillator, the summated biphasic shock vector can be shifted from the anterior part of a patient's body (which usually has a lower impedance) to the posterior part of the patient's body (which usually has a higher impedance) thus achieving a more ideal defibrillation shock vector for a particular patient based on their heart shape, their thorax and lung shape and the particular placement of the subcutaneous defibrillator.

In one embodiment of the disclosed technique, for example with a subcutaneous defibrillator including a plurality of electrodes such as three or more electrodes (not shown), at least one of the electrodes can be disconnected at any given time during the delivery of a shock vector. By disconnecting at least one of the electrodes the energy distribution of the shock vectors to the heart can be directed as desired. Such an embodiment is possible when there are more than two electrodes.

By changing the inversion point timing sequence, the relative amount of energy delivered to each respective shock vector can be dynamically adjusted with the total amount of energy applied by the defibrillator remaining the same yet with its energy distribution being different for each shock vector. Using a numerical example, in a subcutaneous defibrillator which can apply electrical shocks of around 70 joules (i.e., the capacitor can hold sufficient energy to apply 70 joules in a given therapy session of applying electrical shocks), time domain shifting of the inversion point can allow 10 joules to go to one shock vector and 60 joules to the other shock vector, or 35 joules to each shock vector. The energy delivery balance between the two shock vectors can be adjusted such that the DFT is as low as possible while still remaining effective.

As mentioned above, processor 328 may be programmable with options for changing or shifting the inversion point of the applied shock vectors and turning the truncating/summating option of the two shock vectors on or off. Body 322 may include Bluetooth and/or infrared technology (not shown) for enabling a clinician to communicate with processor 328 via software. The software may be a computer application, smartphone application and the like. The decision regarding whether the truncating/summating option of the two shock vectors should be used and to what degree the inversion point should be shifted in time is patient specific and can be determined by the clinician. A number of visits by the patient to the clinician as well as follow-up sessions by the patient after implantation of his/her defibrillator can aid the clinician in determining if the truncating/summating option reduces the DFT and if time domain shifts of the inversion point reduce the number of arrhythmias experienced by the patient.

It is noted that the examples given above of the disclosed technique relate to defibrillators providing two shock vectors, however the disclosed technique can be applied to defibrillators providing three or more shock vectors. According to the disclosed technique, in a defibrillator applying more than two biphasic shock vectors, at least one or more of the biphasic shock vectors can be made uniphasic and terminated at its inversion point, with the remainder of its energy diverted and summated to the other biphasic shock vectors being applied. Furthermore, the inversion point in time of the shock vectors can be shifted for balancing the energy distribution between shock vectors traversing paths of different impedance.

Reference is now made to FIG. 8, which is a method for truncating and summating shock energy between two shock vectors, operative in accordance with another embodiment of the disclosed technique. In a procedure 350, at least two biphasic defibrillating shock vectors are applied via at least two electrode sets until a voltage inversion point. The biphasic shock vectors are applied simultaneously such that each electrode set applies a shock vector. As shown above, a biphasic shock vector includes two phases, a first phase wherein energy is applied via a positive voltage and a second phase wherein energy is applied via a negative voltage. The first phase is separated from the second phase via its voltage inversion point. In this procedure, at least two biphasic shock vectors are applied between a respective at least two sets of electrodes during the first phase of the shock vectors. In a procedure 352, one of the biphasic defibrillating shock vectors is terminated at the voltage inversion point. Thus instead of being a biphasic shock vector, the terminated shock vector is effectively a unipolar shock vector. The termination of the shock vector however does not mean that the energy of the shock vector is lost. That energy is just not applied across the first electrode set. In a procedure 354, the remaining energy of the first biphasic defibrillating shock vector is directed to a second biphasic defibrillating shock vector applied via its second electrode set. The second phase of the second shock vector is thus summated with the energy remaining form the truncated first shock vector, resulting in the second phase of the second shock vector applying an increased amount of energy. As explained above in FIG. 5, since both the first and second biphasic shock vectors are applied simultaneously, terminating the first biphasic shock vector at the end of its first phase and directing the remaining energy to the second biphasic shock vector effectively increases the amount of energy delivered by the second biphasic shock vector. The first biphasic shock vector is thus converted into a uniphasic shock vector whereas the second biphasic shock vector remains biphasic with an increase in energy in its second phase.

In a procedure 356, the voltage inversion point of the biphasic defibrillating shock vectors is modified according to patient and defibrillator characteristics. Depending on the anatomy of the patient and the specific placement of the electrodes of the defibrillator (whether external, intravenously or subcutaneously), the truncating and summating of the energy of the first shock vector to the second shock vector may not be sufficient to balance the energy distribution between the shock vectors to lower the DFT and effectively defibrillate various parts of the heart. In this procedure, the voltage inversion point is shifted in the time domain, either forwards or backwards in time, to transfer either less or more energy from the truncated part of the second phase of the first shock vector to the second phase of the second shock vector. The amount of shifting of the inversion point is dependent on the patient's anatomy and the placement of the electrodes of the defibrillator in or around the patient's heart which can change the impedance of the path the shock vectors take between given sets of electrodes. Modifying the inversion point enables more energy to be delivered to the shock vector having to cross a path of higher impedance. After procedure 356, the method returns to procedure 350.

Procedure 356 can be applied many times until an ideal balance of energy between the two shock vectors is obtained and the DFT for effective defibrillation of a given patient is attained. As mentioned above, even though FIG. 8 and the procedures described therein have been described with reference to two defibrillating shock vectors, the method of the disclosed technique described in FIG. 8 can be used in any type of defibrillator applying at least two shock vectors, for example external, intravenous/transvenous and subcutaneous defibrillators, applying two, three, four or more shock vectors simultaneously.

It will be appreciated by persons skilled in the art that the disclosed technique is not limited to what has been particularly shown and described hereinabove. Rather the scope of the disclosed technique is defined only by the claims, which follow.

1. Method for truncating and summating shock vector energy between at least two shock vectors in a defibrillator, comprising the procedures of:
applying at least two biphasic defibrillating shock vectors simultaneously via at least two electrode sets until a voltage inversion point;
terminating at least a first one of said at least two biphasic defibrillating shock vectors at said voltage inversion point; and
directing a remaining energy of said at least first one of said biphasic defibrillating shock vectors to a second phase of at least a second one of said biphasic defibrillating shock vectors.

2. The method according to claim 1, further comprising the procedure of modifying said voltage inversion point of said at least two biphasic defibrillating shock vectors.

3. The method according to claim 2, wherein said procedure of modifying comprises the sub-procedure of modifying said voltage inversion point according to at least one of patient characteristics and defibrillator characteristics.

4. The method according to claim 3, wherein said patient characteristics comprises an anatomy of a patient.

5. The method according to claim 3, wherein said defibrillator characteristics comprises an actual placement of a plurality of electrodes of said defibrillator in a patient.

6. The method according to claim 2, wherein said procedure of modifying comprises the sub-procedure of modifying said voltage inversion point to achieve energy symmetry between said at least two biphasic defibrillating shock vectors.

7. The method according to claim 1, wherein said at least first one of said at least two biphasic defibrillating shock vectors which is terminated at said voltage inversion point exhibits lower impedance compared to said at least second one of said at least two biphasic defibrillating shock vectors.

8. The method according to claim 1, wherein said defibrillator is selected from the list consisting of:
an external defibrillator;
an intravenous defibrillator;
a transvenous defibrillator; and
a subcutaneous defibrillator.

9. The method according to claim 1, wherein a defibrillating threshold for effective defibrillation of said defibrillator is as low as possible.

10. Subcutaneous defibrillator for truncating and summating at least two biphasic defibrillating shock vectors, comprising:
a body;
a plurality of electrodes, positioned on said body, for applying said at least two biphasic defibrillating shock vectors; and
a plurality of sensors, positioned on said body, for detecting arrhythmias,
said body comprising:
at least one capacitor, for storing charge for providing said at least two biphasic defibrillating shock vectors;
a processor, coupled with said at least one capacitor; and
at least one battery, coupled with said at least one capacitor and said processor, for charging said at least one capacitor and for providing energy to operate said processor,
wherein said plurality of electrodes applies at least a first one of said at least two biphasic defibrillating shock vectors and at least a second one of said at least two biphasic defibrillating shock vectors simultaneously until a voltage inversion point;
wherein said processor terminates said at least first one of said at least two biphasic defibrillating shock vectors at said voltage inversion point; and
wherein said processor directs a remaining energy of said at least first one of said at least two biphasic defibrillating shock vectors to a second phase of said at least second one of said at least two biphasic defibrillating shock vectors.

11. The subcutaneous defibrillator according to claim 10, further comprising a wireless transceiver, coupled with said processor, for programming said processor wirelessly.

12. The subcutaneous defibrillator according to claim 11, wherein said wireless transceiver is selected from the list consisting of:
a Bluetooth® transceiver; and
an infrared transceiver.

13. The subcutaneous defibrillator according to claim 11, wherein said processor can toggle said truncating and summating of said at least two biphasic defibrillating shock vectors on and off via said wireless transceiver.

14. The subcutaneous defibrillator according to claim 13, wherein said subcutaneous defibrillator applies said at least two biphasic defibrillating shock vectors as at least two biphasic defibrillating shock vectors when said processor toggles said truncating and summating off.

15. The subcutaneous defibrillator according to claim 13, wherein said subcutaneous defibrillator applies said at least two biphasic defibrillating shock vectors as at least one truncated uniphasic defibrillating shock vector and at least one summated biphasic defibrillating shock vector when said processor toggles said truncating and summating on.

16. The subcutaneous defibrillator according to claim 11, wherein said processor can modify said voltage inversion point via said wireless transceiver.

17. The subcutaneous defibrillator according to claim 10, wherein said plurality of electrodes comprises at least three electrodes, wherein at least one of said at least three electrodes is disconnected at a given time during the application of said at least two biphasic defibrillating shock vectors.

18. The subcutaneous defibrillator according to claim 10, wherein said at least first one of said at least two biphasic defibrillating shock vectors has a lower impedance compared to said at least second one of said at least two biphasic defibrillating shock vectors having a higher impedance.

19. The subcutaneous defibrillator according to claim 18, wherein said processor truncates said at least first one of said at least two biphasic defibrillating shock vectors having said lower impedance at said voltage inversion point

20. The subcutaneous defibrillator according to claim 18, wherein said processor electronically switches said remaining energy from a first set of said plurality of electrodes applying said at least first one of said at least two biphasic defibrillating shock vectors having said lower impedance to a second set of said plurality of electrodes applying said at least second one of said at least two biphasic defibrillating shock vectors having said higher impedance.

21. Defibrillator for truncating and summating at least two biphasic defibrillating shock vectors, comprising:
a can; and
a plurality of leads, coupled with said can, for detecting arrhythmias;
said can comprising:
at least one capacitor, for storing charge for providing said at least two biphasic defibrillating shock vectors;
a processor, coupled with said at least one capacitor; and
at least one battery, coupled with said at least one capacitor and said processor, for charging said at least one capacitor and for providing energy to operate said processor,
wherein said plurality of leads applies at least a first one of said at least two biphasic defibrillating shock vectors and at least a second one of said at least two biphasic defibrillating shock vectors simultaneously until a voltage inversion point;
wherein said processor terminates said at least first one of said at least two biphasic defibrillating shock vectors at said voltage inversion point; and
wherein said processor directs a remaining energy of said at least first one of said at least two biphasic defibrillating shock vectors to a second phase of said at least second one of said at least two biphasic defibrillating shock vectors.

22. The defibrillator according to claim 21, wherein said defibrillator is selected from the list consisting of:
an external defibrillator;
an intravenous defibrillator; and
a transvenous defibrillator.
